(19) United States
(12) Patent Application Publication
(10) Pub. No.: US 2022/0323437 A1
(43) Pub. Date: Oct. 13, 2022

(54) METHODS FOR TREATING PULMONARY ARTERIAL HYPERTENSION

(71) Applicant: ACTELION PHARMACEUTICALS LTD, Allschwil (CH)

(72) Inventors: Ralph T. PREISS, Allschwil (CH); Shirin BRUDERER, Allschwil (CH); Martine CLOZEL, Allschwil (CH); Natalia Catalina YANNOULIS ARCEDA, Allschwil (CH)

(21) Appl. No.: 17/616,313

(22) PCT Filed: Jun. 10, 2020
(86) PCT No.: PCT/EP2020/066055

§ 371 Date: Dec. 3, 2021

Related U.S. Application Data
(60) Provisional application No. 62/860,121, filed on Jun. 11, 2019.

(51) Int. Cl. A61K 31/4965; A61P 9/12

(52) U.S. Cl. CPC A61K 31/4965; A61P 9/12

(57) ABSTRACT

The present disclosure provides methods of avoiding treatment interruption in a patient receiving an oral dose of selexipag for treating pulmonary arterial hypertension, wherein the patient is temporarily unable to take oral medication. These methods comprise administering to the patient an intravenous (IV) dose of selexipag, and, subsequently, returning to an oral dose of selexipag.

### CROSS-REFERENCE TO RELATED APPLICATIONS

This application claims priority to U.S. Provisional Patent Application No. 62/860,121, filed Jun. 11, 2019, the disclosure of which is incorporated by reference herein.

### FIELD OF THE INVENTION

The present invention relates to methods of avoiding treatment interruption in a patient receiving an oral dose of selexipag for treating pulmonary arterial hypertension.

### BACKGROUND

Selexipag or its active metabolite is known to be useful as a preventive or therapeutic agent for ulcer, digital ulcer, diabetic gangrene, diabetic foot ulcer, pressure ulcer (bedsore), hypertension, pulmonary hypertension, pulmonary arterial hypertension, Fontan disease and pulmonary hypertension associated with Fontan disease, sarcoidosis and pulmonary hypertension associated with sarcoidosis, peripheral circulatory disturbance (e.g., chronic arterial occlusion, intermittent claudication, peripheral embolism, vibration syndrome, Raynaud's disease), connective tissue disease (e.g., systemic lupus erythematosus, scleroderma, mixed connective tissue disease, vasculitic syndrome), reocclusion/restenosis after percutaneous transluminal coronary angioplasty (PTCA), arteriosclerosis, thrombosis (e.g., acute-phase cerebral thrombosis, pulmonary embolism), transient ischemic attack (TIA), diabetic neuropathy, ischemic disorder (e.g., cerebral infarction, myocardial infarction), angina (e.g., stable angina, unstable angina), chronic kidney diseases including glomerulonephritis and diabetic nephropathy at any stage, allergy, bronchial asthma, restenosis after coronary intervention such as atherectomy and stent implantation, thrombocytopenia by dialysis, the diseases in which fibrosis of organs or tissues is involved (e.g., renal diseases such as tubulointerstitial nephritis), respiratory diseases (e.g. (usual) interstitial pneumonia/(idiopathic) pulmonary fibrosis, chronic obstructive pulmonary disease), digestive diseases (e.g., hepatocirrhosis, viral hepatitis, chronic pancreatitis and scirrhous stomachic cancer), cardiovascular diseases (e.g., myocardial fibrosis), bone and articular diseases (e.g., bone marrow fibrosis and rheumatoid arthritis), skin diseases (e.g., cicatrix after operation, scalded cicatrix, keloid, and hypertrophic cicatrix), obstetric diseases (e.g., hysteromyoma), urinary diseases (e.g., prostatic hypertrophy), other diseases (e.g., Alzheimer's disease, sclerosing peritonitis, type I diabetes and organ adhesion after operation)], erectile dysfunction (e.g., diabetic erectile dysfunction, psychogenic erectile dysfunction, psychotic erectile dysfunction, erectile dysfunction associated with chronic renal failure, erectile dysfunction after intrapelvic operation for removing prostata, and vascular erectile dysfunction associated with aging and arteriosclerosis), inflammatory bowel disease (e.g., ulcerative colitis, Crohn's disease, intestinal tuberculosis, ischemic colitis and intestinal ulcer associated with Behcet disease), gastritis, gastric ulcer, ischemic ophthalmopathy (e.g., retinal artery occlusion, retinal vein occlusion, ischemic optic neuropathy), sudden hearing loss, avascular necrosis of bone, intestinal damage caused by administration of a non-steroidal anti-inflammatory agent and symptoms associated with lumbar spinal canal stenosis.

Selexipag was also shown to be beneficial in the treatment of pulmonary arterial hypertension. In a phase III clinical trial, among patients with pulmonary arterial hypertension, the risk of the primary composite end point of death or a complication related to pulmonary arterial hypertension was significantly lower among patients who received selexipag than among those who received placebo. Selexipag received market approval e.g. in the US and is indicated for the treatment of pulmonary arterial hypertension (PAH, WHO Group I) to delay disease progression and reduce the risk of hospitalization for PAH.

So far, standard film-coated tablet formulations of selexipag intended for twice daily oral administration have been used, wherein excipients comprise D-mannitol, corn starch, low substituted hydroxypropylcellulose, hydroxypropylcellulose, and magnesium stearate; and the tablets are film coated with a coating material containing hypromellose, propylene glycol, titanium dioxide, carnauba wax along with mixtures of iron oxides.

Selexipag is thought to function as a prodrug (while retaining some agonistic activity on the IP receptor on its own) which can exert long-lasting selective IP receptor agonist activity of the active metabolite in mammals, especially humans.

Adverse effects associated with PGI2 agonists are also addressed by a particular up-titration schedule. The recommended starting dose of oral selexipag is 200 micrograms given twice daily. The dose is then increased in increments of 200 micrograms twice daily, usually at weekly intervals, to the highest tolerated dose up to 1600 micrograms twice daily. If a patient reaches a dose that cannot be tolerated, the dose should be reduced to the previous tolerated dose.

In addition, there is a need to help avoid treatment interruptions in patients temporarily unable to take oral medication and to maintain the safety, efficacy, and tolerability of the treatment during a temporary switch from an oral dose to another route of administration, such as an intravenous route.

### SUMMARY

The present disclosure provides methods of avoiding treatment interruption in a patient receiving an oral dose of selexipag for treating pulmonary arterial hypertension, wherein the patient is temporarily unable to take oral medication. These methods comprise administering to the patient an intravenous (IV) dose of selexipag, and, subsequently, returning to the same oral dose as prescribed before the IV dose.

The present disclosure also provides methods of selling a drug product comprising selexipag. The method comprises selling the drug product, wherein a drug product label for a reference listed drug for the drug product includes instructions for avoiding treatment interruption in a patient receiving an oral dose of selexipag for treating pulmonary arterial hypertension. In these methods, the patient is temporarily unable to take oral medication and is administered an intravenous (IV) dose of selexipag.

The present disclosure further provides methods of offering for sale a drug product comprising selexipag. The method comprises offering for sale such drug product, wherein a drug product label for a reference listed drug for such drug product includes instructions for avoiding treatment interruption in a patient receiving an oral dose of selexipag for treating pulmonary arterial hypertension. In these methods, the patient is temporarily unable to take oral medication and is administered an IV dose of selexipag.

The present disclosure also provides pharmaceutical drug products comprising a clinically proven safe and clinically proven effective amount of selexipag. The pharmaceutical product is packaged and the package includes a label that identifies selexipag as a regulatory approved chemical entity and includes instructions for avoiding treatment interruption in a patient receiving an oral dose of selexipag for treating pulmonary arterial hypertension, wherein the patient is temporarily unable to take oral medication and is administered an intravenous (IV) dose of selexipag.

### DESCRIPTION OF THE DRAWINGS

FIG. 1 is a timeline of the schedule of visits for the study described in Example 3.

FIG. 2 is a flow diagram of the analysis of PK endpoints for selexipag and its active metabolite, after oral selexipag in Period 1 and after i.v. selexipag in Period 2.

FIGS. 3A and 3B are line graphs showing the PK profiles of oral and intravenous dosing of selexipag and its metabolite, respectively.

### DETAILED DESCRIPTION OF THE PREFERRED EMBODIMENTS

In the present disclosure the singular forms “a”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

### Methods

The present disclosure provides methods of avoiding treatment interruption in a patient receiving an oral dose of selexipag for treating pulmonary arterial hypertension. In these methods, the patient is temporarily unable to take oral medication and, thus, is administered an intravenous (IV) dose of selexipag. In some embodiments, the patient is temporarily unable to take oral medication due to a hospitalization.

The terms “pulmonary arterial hypertension” and “PAH” are interchangeable and define a condition of pulmonary hypertension where the patient has high blood pressure in the lungs. PAH occurs when the very small arteries throughout the lungs narrow in diameter, which increases the resistance to blood flow through the lungs. In some embodiments, the underlying cause of the narrowing is not known, i.e., idiopathic pulmonary hypertension. PAH also is classified into subgroups including (i) familial, or heritable PAH, (ii) PAH caused by drugs or toxins, (iii) PAH associated with other conditions such as connective tissue diseases (scleroderma or lupus), congenital heart problems, high blood pressure in the liver, HIV, infections (schistosomiasis), and sickle cell anemia, (iv) PAH caused by rare blood conditions (pulmonary veno-occlusive disease or pulmonary capillary hemangiomatosis, or (v) PAH in babies (persistent pulmonary hypertension of the newborn).

Prior to being administered intravenous selexipag, the patient desirably will have received an oral dose of selexipag for at least about 28 days prior to administration of the IV dose. This period of time in which the patient has been administered the oral dose of selexipag is referred to herein as the first treatment period. In some embodiments, the patient will have been taking the oral dose of selexipag for at least about 30 days, about 60 days, about 90 days, about 180 days, about 210 days, about 240 days, about 270 days, about 300 days, about 330 days, about 360 days, about 1 year, about 2 years, about 3 years, about 4 years, or about 5 years.

The patient also, desirably, did not receive any prostacyclin or prostacyclin analogs, other than selexipag, for at least about 28 days prior to administration of the intravenous dose. The term “prostacyclin” as used herein refers to any pharmaceutical agent that acts as a prostacyclin pathway agent. The prostacyclin may be an inhaled prostacyclin, an oral prostacyclin, or a parenteral prostacyclin, such as a subcutaneous or intravenous prostacyclin. Non-limiting examples of prostacyclins include epoprostenol (Flolan®, Veletri®), treprostinil (Remodulin®, Tyvaso®, Orenitram®), or iloprost (Ilomedin™, Ventavis®). In some embodiments, the prostacyclin is intravenous epoprostenol, treprostinil, or iloprost. In other embodiments, the prostacyclin is subcutaneous treprostinil. In further embodiments, the prostacyclin is inhaled treprostinil or iloprost. In still other embodiments, the prostacyclin is oral treprostinil.

Prior to administration of the intravenous dose of selexipag, the patient preferably has a systolic blood pressure of less than about 90 mmHg prior to administration of the IV dose.

The oral dose of selexipag, on a daily basis, is at least about 10 μg. In some embodiments, the oral dose of selexipag, on a daily basis, is at least about 100, about 200, about 300, about 400, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, or about 3500 μg.

The daily dose of oral selexipag may be administered once daily, twice daily, or thrice daily, preferably twice daily. In some embodiments, the oral dose is administered twice daily. In other embodiments, the oral dose is administered once before mid-day and once after mid-day.

Desirably, the oral dose of selexipag does not exceed about 1600 μg twice daily, i.e., 3200 μg per day. In some embodiments, the oral dose of selexipag, twice daily, is about 100 to about 3500 μg, about 200 to about 3200, about 200 to about 3000, about 200 to about 2800, about 200 to about 2600, about 200 to about 2400, about 200 to about 2200, about 200 to about 2000, about 200 to about 1800, about 200 to about 1600, about 200 to about 1400, about 200 to about 1200, about 200 to about 1000, about 200 to about 800, about 200 to about 600, about 200 to about 400, about 400 to about 3200, about 400 to about 3000, about 400 to about 2800, about 400 to about 2600, about 400 to about 2400, about 400 to about 2200, about 400 to about 2000, about 400 to about 1800, about 400 to about 1600, about 400 to about 1400, about 400 to about 1200, about 400 to about 1000, about 400 to about 800, about 400 to about 600, about 600 to about 3200, about 600 to about 3000, about 600 to about 2800, about 600 to about 2600, about 600 to about 2400, about 600 to about 2200, about 600 to about 2000, about 600 to about 1800, about 600 to about 1600, about 600 to about 1400, about 600 to about 1200, about 600 to about 1000, about 600 to about 800, about 800 to about 3200, about 800 to about 3000, about 800 to about 2800, about 800 to about 2600, about 800 to about 2400, about 800 to about 2200, about 800 to about 2000, about 800 to about 1800, about 800 to about 1600, about 800 to about 1400, about 800 to about 1200, about 800 to about 1000, about 1000 to about 3200, about 1000 to about 3000, about 1000 to about 2800, about 1000 to about 2600, about 1000 to about 2400, about 1000 to about 2200, about 1000 to about 2000, about 1000 to about 1800, about 1000 to about 1600, about 1000 to about 1400, about 1000 to about 1200, about 1200 to about 3200, about 1200 to about 3000, about 1200 to about 2800, about 1200 to about 2600, about 1200 to about 2400, about 1200 to about 2200, about 1200 to about 2000, about 1200 to about 1800, about 1200 to about 1600, about 1200 to about 1400, about 1400 to about 3200, about 1400 to about 3000, about 1400 to about 2800, about 1400 to about 2600, about 1400 to about 2400, about 1400 to about 2200, about 1400 to about 2000, about 1400 to about 1800, about 1400 to about 1600, about 1600 to about 3200, about 1600 to about 3000, about 1600 to about 2800, about 1600 to about 2600, about 1600 to about 2400, about 1600 to about 2200, about 1600 to about 2000, about 1600 to about 1800, about 1800 to about 3200, about 1800 to about 3000, about 1800 to about 2800, about 1800 to about 2600, about 1800 to about 2400, about 1800 to about 2200, about 1800 to about 2000, about 2000 to about 3200, about 2000 to about 3000, about 2000 to about 2800, about 2000 to about 2600, about 2000 to about 2400, about 2000 to about 2200, about 2200 to about 3200, about 2200 to about 3000, about 2200 to about 2800, about 2200 to about 2600, about 2200 to about 2400, about 2400 to about 3200, about 2400 to about 3000, about 2400 to about 2800, about 2400 to about 2600, about 2600 to about 3200, about 2600 to about 3000, about 2600 to about 2800, about 2800 to about 3200, about 2800 to about 3000, or about 3000 to about 3200 μg. In further embodiments, the oral dose of selexipag, on a twice daily basis, is about 200 to about 1600 μg. In other embodiments, the oral dose of selexipag, on a twice daily basis, is about 200 μg to about 1600 μg. In yet further embodiments, the oral dose of selexipag, on a twice daily basis is about 1200 μg to about 1600 μg twice daily.

Following administration of the oral dose of selexipag, an intravenous dose of selexipag is administered to the patient. This period of time is referred to herein as the second treatment period.

The intravenous dose of selexipag is selected to achieve a comparable exposure to ACT-333679 (active metabolite of selexipag) when compared to the oral dose of selexipag. The term “comparable exposure” as used herein refers to an IV dose that renders a similar plasma exposure to the active metabolite (ACT-333679) as would a corresponding oral dose of selexipag. Guidance in making such a selection may be based on bioavailability studies (see, e.g., Kaufmann P, et al., Eur. J. Clin. Pharmacol. 2017, 73:151-60, which is incorporated herein by reference). Typically, and as shown in the example section, the ratio of the intravenous dose of selexipag to the oral dose of selexipag is greater than 1. In some aspects, the ratio of the intravenous dose of selexipag to the oral dose of selexipag is about 1.125 to about 1.

In some embodiments, the intravenous dose of selexipag, twice daily, is about 112 to about 4000 μg, about 225 to about 3600, about 225 to about 3400, about 225 to about 3150, about 225 to about 3000, about 225 to about 2700, about 225 to about 2500, about 225 to about 2250, about 225 to about 2000, about 225 to about 1800, about 225 to about 1600, about 225 to about 1350, about 225 to about 1125, about 225 to about 900, about 225 to about 675, about 225 to about 450, about 450 to about 3600, about 450 to about 3400, about 450 to about 3150, about 450 to about 3000, about 450 to about 2700, about 450 to about 2500, about 450 to about 2250, about 450 to about 2000, about 450 to about 1800, about 450 to about 1600, about 450 to about 1350, about 450 to about 1125, about 450 to about 900, about 450 to about 675, about 675 to about 3600, about 675 to about 3400, about 675 to about 3150, about 675 to about 3000, about 675 to about 2700, about 675 to about 2500, about 675 to about 2250, about 675 to about 2000, about 675 to about 1800, about 675 to about 1600, about 675 to about 1350, about 675 to about 1125, about 675 to about 900, about 900 to about 3600, about 900 to about 3400, about 900 to about 3150, about 900 to about 3000, about 900 to about 2700, about 900 to about 2500, about 900 to about 2250, about 900 to about 2000, about 900 to about 1800, about 900 to about 1600, about 900 to about 1350, about 900 to about 1125, about 1125 to about 3600, about 1125 to about 3400, about 1125 to about 3150, about 1125 to about 3000, about 1125 to about 2700, about 1125 to about 2500, about 1125 to about 2250, about 1125 to about 2000, about 1125 to about 1800, about 1125 to about 1600, about 1125 to about 1350, about 1350 to about 3600, about 1350 to about 3400, about 1350 to about 3150, about 1350 to about 3000, about 1200 to about 2700, about 1350 to about 2500, about 1350 to about 2250, about 1350 to about 2000, about 1350 to about 1800, about 1350 to about 1600, about 1600 to about 3600, about 1600 to about 3400, about 1600 to about 3150, about 1600 to about 3000, about 1600 to about 2700, about 1600 to about 2500, about 1600 to about 2250, about 1600 to about 2000, about 1600 to about 1800, about 1800 to about 3600, about 1800 to about 3400, about 1800 to about 3150, about 1800 to about 3000, about 1800 to about 2700, about 1800 to about 2500, about 1800 to about 2250, about 1800 to about 2000, about 2000 to about 3600, about 2000 to about 3400, about 2000 to about 3150, about 2000 to about 3000, about 2000 to about 2700, about 2000 to about 2500, about 2000 to about 2250, about 2250 to about 3600, about 2250 to about 3400, about 2250 to about 3150, about 2250 to about 3000, about 2250 to about 2700, about 2250 to about 2500, about 2500 to about 3600, about 2500 to about 3400, about 2500 to about 3150, about 2500 to about 3000, about 2500 to about 2700, about 2700 to about 3600, about 2700 to about 3400, about 2700 to about 3150, about 2700 to about 3000, about 3000 to about 3600, about 3000 to about 3400, about 3000 to about 3150, about 3150 to about 3600, about 3150 to about 3400, or about 3400 to about 3600 μg. In further embodiments, the intravenous dose of selexipag, on a twice daily basis, is about 225 to about 1800 μg. In other embodiments, the intravenous dose of selexipag, on a twice daily basis, is about 225 μg to about 1800 μg. In yet further embodiments, the intravenous dose of selexipag, on a twice daily basis is about 1000 μg to about 1800 μg twice daily.

The second treatment period comprises at least one infusion of the intravenous dose of selexipag. In some embodiments, the second treatment period comprises two infusions of the intravenous dose of selexipag. In other embodiments, the second period comprises three infusions of the IV dose. In further embodiments, the second treatment period comprises one to three infusions of the intravenous dose of selexipag.

Each infusion is administered to the patient over a period of time as determined by the attending physician. In some embodiments, each infusion is at least about 60 minutes in duration. In other embodiments, each infusion is about 60 to about 120 minutes in duration. In further embodiments, each infusion is from about 80 to about 90 minutes in duration. In yet other embodiments, each infusion is about 80 minutes in duration. In still further embodiments, each infusion is about 90 minutes in duration.

The infusions may be administered on separate days or on the same day. In some embodiments, only one infusion is administered per day. In other embodiments, two infusions are administered in one day. In further embodiments, two infusions are administered in one day, once before mid-day and once after mid-day. In yet other embodiments, a third infusion is administered on the next day following the second infusion.

The term “mid-day” as used herein refers to the period of time between ante meridiem (a.m.; before mid-day and post meridiem (p.m.; after midday).

Following administration of the intravenous dose of selexipag, the patient resumes treatment with oral selexipag. The resumption of the oral dose of selexipag is referred to herein as the third treatment period. In some embodiments, the patient resumes treatment with the same oral dose as prescribed before the intravenous dose administration. In other embodiments, the third period comprises resumption of the oral dose on the same day of, and following, the third infusion of intravenous selexipag. In other embodiments, the patient resumes treatment with a different oral dose as that prescribed before the intravenous dose.

In some preferred embodiments, the first period of treatment comprises the administration of the oral dose of selexipag, a second period comprises the administration of the IV dose of selexipag, and a third period comprises resumption of an oral dose of selexipag. In other preferred embodiments, the first period of treatment comprises the administration of the oral dose, a second period comprises the administration of the IV dose, and a third period comprises resumption of the same oral dose as administered in the first period.

The methods described herein desirably result in an area under the plasma-concentration time curve during a dose interval at steady state (AUC_{τ, ss}) of selexipag that is about two times greater in the second period (following IV administration) than in the first period (oral administration). The methods described herein may also or alternatively result in an area under the plasma-concentration time curve during a dose interval at steady state of the active metabolite (ACT-333679) that is comparable after administration of oral selexipag (first period) and IV selexipag (second period).

The methods herein may further result in a time to reach maximum plasma concentration that is comparable between the oral administration period (first period) and the IV administration period (second period).

As used herein, unless otherwise noted, the term “selexipag” refers to 2-{4-[(5,6-diphenylpyrazin-2-yl)(propan-2-yl)amino]butoxy}-N-(methanesulfonyl)acetamide of formula (I).

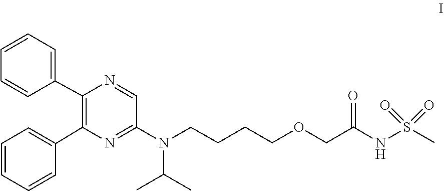 [SMILES: CC(C)N(CCCCOCC(=O)NS(C)(=O)=O)c1cnc(-c2ccccc2)c(-c2ccccc2)n1]

As used herein, “selexipag” also refers to amorphous or crystalline forms of selexipag, such as polymorphs thereof. In some embodiments, the selexipag is a crystalline form, such as a polymorph. In other embodiments, the selexipag is an amorphous form. In other embodiments, the selexipag is the Form I as described in U.S. Pat. Nos. 8,791,122 and 9,284,280, Form II as described in U.S. Pat. No. 9,340,516, or Form III as described in U.S. Pat. No. 9,440,931, all of which are incorporated by reference herein. The crystallinity may be determined by those skilled in the art using one or more techniques such as, e.g., single crystal x-ray diffraction, powder x-ray diffraction, differential scanning calorimetry, melting point, among others.

“Selexipag” as used herein includes anhydrous or hydrates thereof. In certain embodiments, the selexipag is an anhydrous form. In other embodiments, the selexipag is a hydrate thereof.

“Selexipag” as used herein further refers to solvates thereof. Such solvates include a molecule of a solvent bound through intermolecular forces or chemical bonds to one or more locations of the selexipag molecule.

The term “selexipag” may also include pharmaceutically acceptable salts thereof, which may readily be selected by those skilled in the art. A “pharmaceutically acceptable salt” is intended to mean a salt of selexipag that is non-toxic, biologically tolerable, or otherwise biologically suitable for administration to the subject. See, e.g., Berge, “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002, which are incorporated herein by reference. Selexipag can be used in the form of a free base or acid, but can also be used after forming into a pharmaceutically acceptable salt by a known method. When the selexipag is basic, examples of “salt” include salts of inorganic acids such as hydrochloric acid, sulfuric acid, nitric acid, phosphoric acid, hydrofluoric acid and hydrobromic acid, and salts of organic acids such as acetic acid, tartaric acid, lactic acid, citric acid, fumaric acid, maleic acid, succinic acid, methanesulfonic acid, ethanesulfonic acid, benzenesulfonic acid, p-toluenesulfonic acid, naphthalenesulfonic acid and camphorsulfonic acid. When the selexipag is acidic, examples of “salt” include alkali metal salts such as sodium salt and potassium salt, and alkali earth metal salts such as calcium salt.

Geometrical isomers (Z form and E form) of selexipag or mixtures thereof are also contemplated.

Selexipag is commercially available as understood to those skilled in the art. See, e.g., U.S. Pat. No. 7,205,302, which is incorporated by reference herein. For example, selexipag is available as Uptravi® and also is known as ACT-293987, NS-304, or JNJ-678896049. Selexipag is an agonist of the prostacyclin receptor and may be prepared according to a process as disclosed in U.S. Pat. No. 7,205,302.

The present disclosure also contemplates the administration of selexipag metabolites. Desirably, the selexipag metabolite is metabolically active compound. Thus, in certain embodiments, the selexipag metabolite is of formula M1. M1 is also known under the code name ACT-333679 or MRE-269.

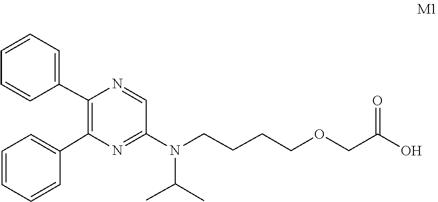 [SMILES: CC(C)N(CCCCOCC(=O)O)c1cnc(-c2ccccc2)c(-c2ccccc2)n1]

As used herein, unless otherwise noted, the terms “treating”, “treatment” and the like, shall include the management and care of a patient for the purpose of combating a disease, condition, or disorder. The terms “treating” and “treatment” also include the administration of the compounds or pharmaceutical compositions as described herein to (a) alleviate one or more symptoms or complications of the disease, condition or disorder; (b) prevent the onset of one or more symptoms or complications of the disease, condition or disorder; and/or (c) eliminate one or more symptoms or complications of the disease, condition, or disorder.

As used herein, unless otherwise noted, the terms “preventing”, “prevention” and the like, shall include (a) reducing the frequency of one or more symptoms; (b) reducing the severity of one or more symptoms; (c) delaying, slowing or avoiding of the development of additional symptoms; and/or (d) slowing, or avoiding the development of the disorder or condition to a later stage or more serious form.

One skilled in the art will recognize that, wherein the present disclosure is directed to methods of prevention, a patient in need thereof shall include any patient or patient who has experienced or exhibited at least one symptom of the disorder, disease or condition to be prevented. Further, a patient in need thereof may additionally be a patient who has not exhibited any symptoms of the disorder, disease or condition to be prevented, but who has been deemed by a physician, clinician or other medical profession to be at risk of developing said disorder, disease or condition. For example, the patient may be deemed at risk of developing a disorder, disease or condition (and therefore in need of prevention or preventive treatment) as a consequence of the patient's medical history, including, but not limited to, family history, pre-disposition, co-existing (comorbid) disorders or conditions, genetic testing, and the like.

The terms “subject” and “patient” are interchangeably used herein to refer to a human, who has been the object of treatment, observation or experiment. Preferably, the patient has experienced and/or exhibited at least one symptom of the disease or disorder to be treated and/or prevented.

In the methods described herein, the therapeutically effective amount of selexipag is safe, effective, or safe and effective. As used herein, unless otherwise noted, the term “safe” shall mean without undue adverse side effects (such as toxicity, irritation, or allergic response), commensurate with a reasonable benefit/risk ratio when used in the manner of this invention. Similarly, unless otherwise noted, the term “effective” means the efficacy of treatment has been demonstrated for the treatment of patients with pulmonary arterial hypertension when dosed in a therapeutically effective dose. In certain embodiments, the methods described herein are safe. In other embodiments, the methods described herein are effective. In further embodiments, the methods described herein are safe and effective. In yet other embodiments, the therapeutically effective amount of selexipag is safe. In still further embodiments, the therapeutically effective amount of selexipag is effective. In other embodiments, the therapeutically effective amount of selexipag is safe and effective.

As used herein, unless otherwise noted, the term “clinically proven” (used independently or to modify the terms “safe” and/or “effective”) shall mean that proof has been proven by a Phase III or IV clinical trial that are sufficient to meet approval standards of U.S. Food and Drug Administration or similar study for market authorization by EMEA. Preferably, an adequately sized, randomized, double-blinded controlled study is used to clinically prove the effects of selexipag as compared to a placebo with the patient's condition assessed by techniques described herein.

As used herein, unless otherwise noted, the term “clinically proven effective” means the efficacy of treatment has been proven by a Phase III or IV clinical trial as statistically significant i.e., the results of the clinical trial are not likely to be due to chance with an alpha level less than 0.05 or the clinical efficacy results are sufficient to meet approval standards of U.S. Food and Drug Administration or similar study for market authorization by EMEA. For example, selexipag was clinically proven effective for the treatment of patients with pulmonary arterial hypertension in a therapeutically effective dose as described herein, and as specifically set forth in the examples.

As used herein, unless otherwise noted, the term “clinically proven safe” means the safety of treatment has been proven by a Phase III or IV clinical trial by analysis of the trial data and results establishing that the treatment is without undue adverse side effects and commensurate with the statistically significant clinical benefit (e.g., efficacy) sufficient to meet approval standards of U.S. Food and Drug Administration or similar study for market authorization by Europe, the Middle East, and Africa (EMEA). For example, selexipag was clinically proven safe for the treatment of patients with pulmonary arterial hypertension when dosed in a therapeutically effective dose as described herein, and as specifically set forth in the examples.

In certain aspects, methods of selling a drug product comprising selexipag are also provided. The terms “sale” or “selling” as used herein refers to transferring a drug product, e.g., a pharmaceutical composition or a dosage form, from a seller to a buyer. Thus, the methods include selling a drug product comprising selexipag, wherein the method comprises selling the drug product. In some embodiments, a drug product label for a reference listed drug for the drug product includes instructions for avoiding treatment interruption in a patient receiving an oral dose of selexipag for treating pulmonary arterial hypertension, wherein the patient is temporarily unable to take oral medication, comprising administering to the patient an intravenous (IV) dose of selexipag. The methods also include offering for sale a drug product comprising selexipag. The term “offering for sale,” as used herein, refers to the proposal of a sale by a seller to a buyer for a drug product, e.g., a pharmaceutical composition or a dosage form. These methods comprise offering the drug product for sale.

In some embodiments, the present disclosure provides pharmaceutical drug products comprising a clinically proven safe and clinically proven effective amount of selexipag, wherein the pharmaceutical product is packaged and wherein the package includes a label that identifies selexipag as a regulatory approved chemical entity and includes instructions for avoiding treatment interruption in a patient receiving an oral dose of selexipag for treating pulmonary arterial hypertension, wherein the patient is temporarily unable to take oral medication, comprising administering to the patient an intravenous (IV) dose of selexipag.

The term “drug product” refers to a product that contains an active pharmaceutical ingredient that has been approved for marketing by a governmental authority, e.g., the Food and Drug Administration or the similar authority in other countries. In some embodiments, the drug product comprises selexipag.

Similarly, “label” or “drug product label” refers to information provided to a patient which provides relevant information regarding the drug product. Such information includes, without limitation, one or more of the description of the drug, clinical pharmacology, indications (uses for the drug product), contraindication (who should not take the drug product), warnings, precautions, adverse events (side effects), drug abuse and dependence, dosage and administration, use in pregnancy, use in nursing mothers, use in children and older patients, how the drug is supplied, safety information for the patient, or any combination thereof. In certain embodiments, the label or drug product label provides an instructions for avoiding treatment interruption in a patient receiving an oral dose of selexipag for treating pulmonary arterial hypertension, wherein the patient is temporarily unable to take oral medication, comprising administering to the patient an intravenous (IV) dose of selexipag. In further embodiments, the label or drug product label identifies selexipag as a regulatory approved chemical entity. In still other embodiments, the label comprises an instruction for achieving comparable exposure to ACT-333679 between the oral dose and IV dose of selexipag. In yet further embodiments, the label includes data comprising area under the plasma-concentration time curve during a dose interval at steady state and time to reach maximum plasma concentration at steady state of selexipag and ACT-333679 based on the oral dose and IV dose of selexipag.

The term “reference listed drug” or “RLD” as used herein refers to a drug product to which new generic versions are compared to show that they are bioequivalent. It is also a medicinal product that has been granted marketing authorization by a member state of the European Union or by the Commission on the basis of a completed dossier, i.e., with the submission of quality, pre-clinical and clinical data in accordance with Articles 8(3), 10a, 10b or 10c of Directive 2001/83/EC and to which the application for marketing authorization for a generic/hybrid medicinal product refers, by demonstration of bioequivalence, usually through the submission of the appropriate bioavailability studies.

In certain embodiments, the drug product is an ANDA drug product, a supplemental New Drug Application drug product, or a 505(b)(2) drug product. In the United States, a company seeking approval to market a generic equivalent must refer to the RLD in its Abbreviated New Drug Application (ANDA). For example, an ANDA applicant relies on the FDA's finding that a previously approved drug product, i.e., the RLD, is safe and effective, and must demonstrate, among other things, that the proposed generic drug product is the same as the RLD in certain ways. Specifically, with limited exceptions, a drug product for which an ANDA is submitted must have, among other things, the same active ingredient(s), conditions of use, route of administration, dosage form, strength, and (with certain permissible differences) labeling as the RLD. The RLD is the listed drug to which the ANDA applicant must show its proposed ANDA drug product is the same with respect to active ingredient(s), dosage form, route of administration, strength, labeling and conditions of use, among other characteristics. In the electronic Orange Book, there is a column for RLDs and a column for reference standards. In the printed version of the Orange Book, the RLDs and reference standards are identified by specific symbol.

In Europe, Applicants identify in the application form for its generic/hybrid medicinal product, which is the same as an ANDA or supplemental NDA (SNDA) drug product, the reference medicinal product (product name, strength, pharmaceutical form, marketing authorization holder (MAH, first authorization, Member State/Community), which is synonymous with a RLD, as follows:
1. The medicinal product that is or has been authorized in the European Economic Area (EEA), used as the basis for demonstrating that the data protection period defined in the European pharmaceutical legislation has expired. This reference medicinal product, identified for the purpose of calculating expiry of the period of data protection, may be for a different strength, pharmaceutical form, administration route or presentation than the generic/hybrid medicinal product.
2. The medicinal product, the dossier of which is cross-referred to in the generic/hybrid application (product name, strength, pharmaceutical form, MAH, marketing authorization number). This reference medicinal product may have been authorized through separate procedures and under a different name than the reference medicinal product identified for the purpose of calculating expiry of the period of data protection. The product information of this reference medicinal product will, in principle, serve as the basis for the product information claimed for the generic/hybrid medicinal product.
3. The medicinal product (product name, strength, pharmaceutical form, MAH, Member State of source) used for the bioequivalence study(ies) (where applicable).

The different abbreviated approval pathways for drug products under the Food, Drug, and Cosmetics (FD&C) Act are the abbreviated approval pathways described in sections 505(j) and 505(b)(2) of the FD&C Act (21 U.S.C. 355(j) and 21 U.S.C. 355(b)(2), respectively).

According to the FDA (“Determining Whether to Submit an ANDA or a 505(b)(2) Application Guidance for Industry,” U.S. Department of Health and Human Services, October 2017, pp. 1-14, the contents of which is incorporated herein by reference), NDAs and ANDAs can be divided into the following four categories:
(1) A “stand-alone NDA” is an application submitted under section 505(b)(1) and approved under section 505(c) of the FD&C Act that contains full reports of investigations of safety and effectiveness that were conducted by or for the applicant or for which the applicant has a right of reference or use.
(2) A section 505(b)(2) application is an NDA submitted under section 505(b)(1) and approved under section 505(c) of the FD&C Act that contains full reports of investigations of safety and effectiveness, where at least some of the information required for approval comes from studies not conducted by or for the applicant and for which the applicant has not obtained a right of reference or use.
(3) An ANDA is an application for a duplicate of a previously approved drug product that was submitted and approved under section 505(j) of the FD&C Act. An ANDA relies on the FDA's finding that the previously approved drug product, i.e., the reference listed drug (RLD), is safe and effective. An ANDA generally must contain information to show that the proposed generic product (a) is the same as the RLD with respect to the active ingredient(s), conditions of use, route of administration, dosage form, strength, and labeling (with certain permissible differences) and (b) is bioequivalent to the RLD. An ANDA may not be submitted if studies are necessary to establish the safety and effectiveness of the proposed product.
(4) A petitioned ANDA is a type of ANDA for a drug product that differs from the RLD in its dosage form, route of administration, strength, or active ingredient (in a product with more than one active ingredient) and for which FDA has determined, in response to a petition submitted under section 505(j)(2)(C) of the FD&C Act (suitability petition), that studies are not necessary to establish the safety and effectiveness of the proposed drug product.

A scientific premise underlying the Hatch-Waxman Act is that a drug product approved in an ANDA under section 505(j) of the FD&C Act is presumed to be therapeutically equivalent to its RLD. Products classified as therapeutically equivalent can be substituted with the full expectation that the substituted product will produce the same clinical effect and safety profile as the prescribed product when administered to patients under the conditions specified in the labeling. In contrast to an ANDA, a section 505(b)(2) application allows greater flexibility as to the characteristics of the proposed product. A section 505(b)(2) application will not necessarily be rated therapeutically equivalent to the listed drug it references upon approval

Thus, the methods of avoiding treatment interruption in a patient receiving an oral dose of selexipag for treating pulmonary arterial hypertension, wherein the patient is temporarily unable to take oral medication, include administering to the patient a drug product comprising an intravenous (IV) dose of selexipag.

The methods may also comprise, consist of, or consist essentially of placing selexipag into the stream of commerce. In certain embodiments, the selexipag includes a package insert that contains instructions for avoiding treatment interruption in a patient receiving an oral dose of selexipag for treating pulmonary arterial hypertension, wherein the patient is temporarily unable to take oral medication, comprising administering to the patient an intravenous (IV) dose of selexipag.

In further aspects, described herein are methods of selling a pharmaceutical composition containing selexipag comprising, consisting of, or consisting essentially of placing the pharmaceutical composition into the stream of commerce. In certain embodiments, the pharmaceutical composition includes a package insert that contains instructions for avoiding treatment interruption in a patient receiving an oral dose of selexipag for treating pulmonary arterial hypertension, wherein the patient is temporarily unable to take oral medication, comprising administering to the patient an intravenous (IV) dose of selexipag.

In still further aspects, described herein are methods of offering for sale selexipag comprising, consisting of, or consisting essentially of offering to place the selexipag into the stream of commerce. In certain embodiments, the selexipag includes a package insert that contains instructions for avoiding treatment interruption in a patient receiving an oral dose of selexipag for treating pulmonary arterial hypertension, wherein the patient is temporarily unable to take oral medication, comprising administering to the patient an intravenous (IV) dose of selexipag.

### Formulations/Compositions

Pharmaceutical compositions containing selexipag as the active ingredient can be prepared by intimately mixing the compound or compounds with a pharmaceutical carrier according to conventional pharmaceutical compounding techniques. As used herein, the terms “composition” and “formulation” are used interchangeably and encompass a product comprising the specified ingredients in the specified amounts, as well as any product, such as a pharmaceutical product, which results, directly or indirectly, from combinations of the specified ingredients in the specified amounts. A summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Selexipag may be administered to a patient neat or in a mixture with a pharmaceutically acceptable non-toxic inert carrier, for example, as a pharmaceutical composition containing the compound at a level of 0.1% to 99.5 wt %, preferably 0.5% to 90%, based on the total weight of the composition. As a carrier, one or more of auxiliary agents for formulations such as solid, semi-solid and liquid diluent, filler and other auxiliary agents for drug formulations may be used. It is desirable that a pharmaceutical composition is administered as a unit dosage form.

The pharmaceutical compositions may be administered by a number of routes as determined by those skilled in the art. Preferably, the pharmaceutical compositions are administered by route that is suitable for selexipag. In some embodiments, the pharmaceutical compositions are administered orally, parenterally, or any combination thereof. In other embodiments, the pharmaceutical compositions are administered orally. In further embodiments, the pharmaceutical compositions are administered parenterally such as intravenously.

In some embodiments, the pharmaceutical compositions are administered as injections or infusions such as intravenous injections. For intravenous administration, the pharmaceutical composition or pharmaceutical product is a sterile solution. Injectable suspensions or solutions may be prepared utilizing aqueous carriers along with appropriate additives. For intravenous administration, the carrier will usually consist of sterile water and other ingredients which increase solubility or preservation. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin. Isotonic preparations which may contain suitable preservatives are employed when intravenous administration is desired. In some embodiments, the carrier used in intravenous formulations comprises sterile water.

For oral administration, the preparation may be solid or liquid. Preferably, the oral form of selexipag as described herein is a solid. Examples of solid formulations include, for example, pastilles, thin films, pastes, lozenges, granules, powders, capsules, pills such as caplets, gelcaps, tablets, and capsules (each including immediate release, timed release and sustained release pills). Preferably, the oral compositions are administered as tablets, i.e., desirably, the pharmaceutical product comprises a tablet. If desired, tablets or caplets may be sugar coated or enteric coated by standard techniques or otherwise compounded to provide a dosage form affording the advantage of prolonged action. For preparing solid compositions such as tablets, the principal active ingredient (e.g., selexipag) is mixed with a pharmaceutical carrier/additive such as starches, sweeteners such as sugars, diluents, coloring agents, granulating agents, preservatives, lubricants, flavoring agents, binders, disintegrating agents and the like. For tablets, conventional tableting ingredients such as corn starch, lactose, sucrose, sorbitol, talc, stearic acid, magnesium stearate, dicalcium phosphate or gums, and other pharmaceutical diluents, e.g., water, ethanol, glycerol, or the like, may be used. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrating agents include, without limitation, starch, methyl cellulose, agar, bentonite, xanthan gum and the like. The liquid forms in which the compositions of the present disclosure may be incorporated for administration by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. The liquid oral preparations contain selexipag and one or more of suitable carriers/additives such as water, glycols, oils, alcohols, flavoring agents, preservatives, stabilizers, coloring agents and the like.

To prepare pharmaceutical compositions of the present disclosure, selexipag, as the active ingredient, may be intimately admixed with a pharmaceutical carrier according to conventional pharmaceutical compounding techniques, which carrier may take a wide variety of forms depending of the form of preparation desired for administration (e.g., oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain, the disclosure of which is hereby incorporated by reference.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc., the disclosures of which are hereby incorporated by reference.

The preparation of selexipag is described in WO2002/088084 (incorporated herein by reference). The preparation of polymorphic forms, i.e. the crystalline forms I, II, and III of the free base is disclosed in WO2010/150865 (incorporated herein by reference); polymorphic forms of pharmaceutically acceptable salts are disclosed in WO2011/024874 (incorporated herein by reference).

The present disclosure also includes at least the following Aspects:

1. A method of selling a drug product comprising selexipag, said method comprising selling the drug product, wherein a drug product label for a reference listed drug for the drug product includes instructions for avoiding treatment interruption in a patient receiving an oral dose of selexipag for treating pulmonary arterial hypertension, wherein the patient is temporarily unable to take oral medication, comprising administering to the patient an intravenous (IV) dose of selexipag.

2. A method of offering for sale a drug product comprising selexipag, said method comprising offering for sale such drug product, wherein a drug product label for a reference listed drug for such drug product includes instructions for avoiding treatment interruption in a patient receiving an oral dose of selexipag for treating pulmonary arterial hypertension, wherein the patient is temporarily unable to take oral medication, comprising administering to the patient an IV dose of selexipag.

3. The method of Aspect 1 or 2, wherein the drug product is an ANDA drug product, a supplemental New Drug Application drug product, or a 505(b)(2) drug product.

4. The method of any one of the preceding Aspects, wherein the label provides an instruction for achieving comparable exposure to ACT-333679 between the oral dose and IV dose of selexipag.

5. The method of Aspect 4, wherein the label includes data comprising area under the plasma-concentration time curve during a dose interval at steady state and time to reach maximum plasma concentration at steady state of selexipag and ACT-333679 based on the oral dose and IV dose of selexipag.

6. A pharmaceutical drug product comprising a clinically proven safe and clinically proven effective amount of selexipag, wherein the pharmaceutical product is packaged and wherein the package includes a label that identifies selexipag as a regulatory approved chemical entity and includes instructions for avoiding treatment interruption in a patient receiving an oral dose of selexipag for treating pulmonary arterial hypertension, wherein the patient is temporarily unable to take oral medication, comprising administering to the patient an intravenous (IV) dose of selexipag.

7. The pharmaceutical product of Aspect 6, wherein the label provides an instruction for achieving comparable exposure to ACT-333679 between the oral dose and IV dose of selexipag.

8. The pharmaceutical product of Aspect 7, wherein the label includes data comprising area under the plasma-concentration time curve during a dose interval at steady state and time to reach maximum plasma concentration at steady state of selexipag and ACT-333679 based on the oral dose and IV dose of selexipag.

Abbreviations: bid=twice-daily; CHD=congenital heart disease; CTD=connective tissue disorder; ERA=endothelin receptor antagonist; PDE-5=phosphodiesterase-5; sGC=soluble guanylate cyclase.

### EXAMPLES

### Example 1: Oral Composition

Oral selexipag is supplied as round, film-coated tablets in different strengths: 200, 400, 600, 800, 1000, 1200, 1400, and 1600 μg strength.

The oral selexipag compositions are given in Table 1 and Table 2.

TABLE 1
Composition of selexipag film-coated tablets (200-800 μg)

| | Selexipag film-coated tablet | | | |
|---|---|---|---|---|
| | 200 μg | 400 μg | 600 μg | 800 μg |
| Ingredients | Amount (mg) | | | |
| Selexipag | 0.2 | 0.4 | 0.6 | 0.8 |
| Mannitol | 72.6 | 72.4 | 72.2 | 72.0 |
| Maize starch | 48.0 | | | |
| Low substituted hydroxypropylcellulose | 6.8 | | | |
| Hydroxypropylcellulose | 5.4 | | | |
| Magnesium stearate | 2.0 | | | |
| Core tablet weight | 135.0 | | | |
| Hypromellose | 3.8000 | | | |
| Propylene glycol | 0.7000 | | | |
| Titanium dioxide | 0.4505 | 0.3500 | 0.4880 | 0.1500 |
| Iron oxide red | — | 0.1500 | 0.0050 | — |
| Iron oxide black | — | — | 0.0070 | 0.1000 |
| Iron oxide yellow | 0.0495 | — | — | 0.2500 |

| | |
|---|---|
| Carnauba wax | 0.040 |
| Coating weight | 5.0 |
| Total weight of film-coated tablet | 140.0 mg |

TABLE 2
Composition of selexipag film-coated tablets (1000-1600 μg)

| | Selexipag film-coated tablet | | | |
|---|---|---|---|---|
| | 1000 μg | 1200 μg | 1400 μg | 1600 μg |
| Ingredients | Amount (mg) | | | |
| Selexipag | 1.0 | 1.2 | 1.4 | 1.6 |
| Mannitol | 71.8 | 71.6 | 71.4 | 71.2 |
| Maize starch | 48.0 | | | |
| Low substituted | 6.8 | | | |
| hydroxypropylcellulose | | | | |
| Hydroxypropylcellulose | 5.4 | | | |
| Magnesium stearate | 2.0 | | | |
| Core tablet weight | 135.0 | | | |
| Hypromellose | 3.8000 | | | |
| Propylene glycol | 0.7000 | | | |
| Titanium dioxide | 0.4310 | 0.4640 | 0.2500 | 0.1500 |
| Iron oxide red | 0.0195 | 0.0150 | — | 0.1250 |
| Iron oxide black | — | 0.0210 mg | — | 0.1250 |
| Iron oxide yellow | 0.0495 | — | 0.2500 | 0.1000 |

| | |
|---|---|
| Carnauba wax | 0.040 |
| Coating weight | 5.0 |
| Total weight of | 140.0 mg |
| film-coated tablet | |

### Example 2: Intravenous Formulation

The selexipag IV formulation referenced in this example contains the components described in Table 3.

TABLE 3

| Component | Label Claim (mg) | Amount per vial (mg/vial)* |
|---|---|---|
| Selexipag | 1.80 | 1.90 |
| Glycine | 180 | 190 |
| Phosphoric Acid | 3.52 | 3.72 |
| Polysorbate 20 | 10.8 | 11.4 |
| NaOH | 1.71 | 1.8 |
| | | Adjusted to pH 7.5 ± 0.2 |
| Water for injection | — | Adjusted to 3800 |

*A fill volume overage of 5% is applied in order to ensure the withdraw of the full label dose

### Example 3

### A. Objectives

The primary objective of the study was to assess whether temporary switching from a stable oral dose of selexipag to an intravenous (i.v.) dose of selexipag provides comparable exposure to active metabolite ACT-333679 and switching back to the initial oral dose of selexipag was safe and well tolerated in subjects with stable PAH.

The first secondary objective was to evaluate the safety and tolerability of selexipag during each study period. The second secondary objective was to evaluate the pharmacokinetics (PK) of selexipag and its active metabolite, ACT-333679, at stable oral dose at steady state and after the switch from oral to i.v. selexipag in subjects with stable PAH.

### B. Study Design

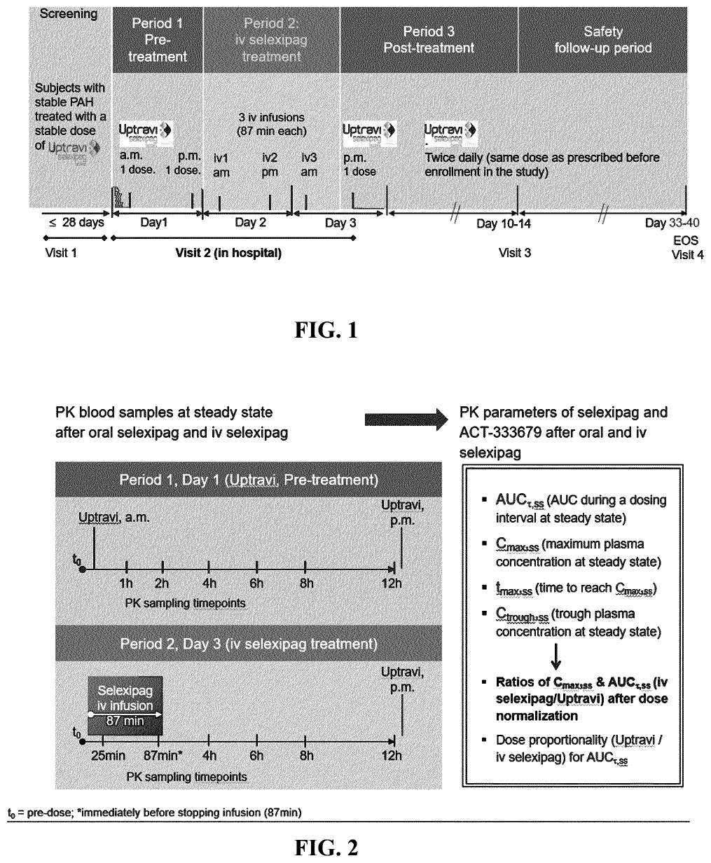

This was a prospective, multicenter, open-label, single-sequence, cross-over, Phase 3 study. Based on the dose of selexipag received at study entry, subjects were allocated to one of the following groups, selexipag 200-1000 μg b.i.d. or selexipag 1200-1600 μg b.i.d. See, FIG. 1. The study comprised the following periods:

(i) Screening period: Screening assessments were performed between Day-28 and Day-1 (Visit 1). The period started with the signing of the ICF and ended on Day 1 at Visit 2, prior to start of Period 1.

(ii) Treatment and observation period: The treatment and observation period included the following consecutive periods:
Period 1 included oral selexipag pre-treatment period for 1 day, in-hospital. This period started with intake of the morning dose of oral selexipag on Day 1 at Visit 2 and ended the following day, before initiation of the first infusion of i.v. selexipag.
Period 2 included i.v. selexipag treatment period for 36 hours [3 infusions], in-hospital. This period started in the morning of Day 2 at Visit 2 with the start of the first infusion of i.v. selexipag and ended in the evening of Day 3 at Visit 2 before the evening administration of oral selexipag. Administration of oral selexipag was interrupted during the i.v. selexipag treatment period.
Period 3 included oral selexipag post-treatment period for 7 to 11 day. This period started in the evening of Day 3 at Visit 2 with the administration of oral selexipag and ended 7-11 days later, at Visit 3.

(c) The safety follow-up period started at the end of Visit 3 and ended 30-37 days after the last administration of i.v. selexipag with an End-of-Study (EOS) telephone call (Visit 4).

### C. Number of Subjects

A total of 20 subjects were enrolled in the study. The Safety analysis set, i.v. Safety analysis set, and PK analysis set were identical and included all 20 subjects enrolled in the study.

### D. Study Population

The study enrolled male and female subjects diagnosed with Group 1 PAH, aged 18-75 years, who were prescribed oral selexipag as part of their standard treatment for PAH. Eligible subjects were required to be on a stable dose of oral selexipag for at least 28 days prior to enrollment.

Subjects were in WHO functional class (FC) I-III. Concomitant treatment with diuretics or other PAH-specific medications that were not agonists of the IP receptor (i.e., ERA, PDE-5 inhibitors, or sGC stimulators) was allowed if subjects had been on a stable dose for a minimum of 28 days. Subjects with significant cardiovascular, hepatic, or renal medical conditions were excluded.

### E. Treatment

Selexipag was provided as a dry powder to be reconstituted as an injectable solution for i.v. administration. Before administration, selexipag dry powder was to be reconstituted and diluted with sterile 0.9% NaCl solution. Each subject received an i.v. selexipag dose that corresponded to the subject's individual stable dose of oral selexipag. The i.v. to oral dose ratio was 1.125 and was designed to obtain comparable exposure to the active metabolite (ACT-333679) between oral selexipag and iv selexipag. See, Table 4.

TABLE 4

| Selexipag | Selexipag i.v. |
|---|---|
| oral dose (μg) | dose (μg) |
| 200 | 225 |
| 400 | 450 |
| 600 | 675 |
| 800 | 900 |
| 1000 | 1125 |
| 1200 | 1350 |
| 1400 | 1575 |
| 1600 | 1800 |

The intravenous infusion took place over a 87-minute period (2 infusions on Day 2 and 1 infusion on Day 3)

### F. Medicinal Products

Prescribed oral selexipag tablets in the dose range 200-1600 μg b.i.d. oral selexipag was to be temporarily interrupted for 36 hours during the administration of study treatment (i.v. selexipag).

### G. Criteria for Evaluation

(i) Pharmacokinetics

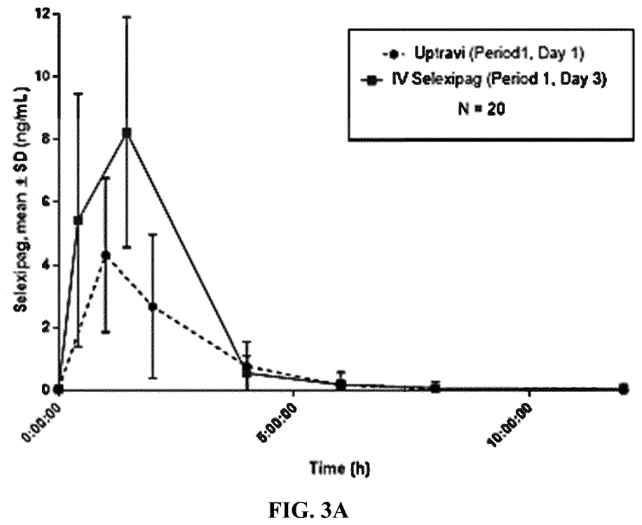

The following PK endpoints were analyzed for selexipag and its active metabolite, ACT-333679, after oral selexipag in Period 1 and after i.v. selexipag administration in Period 2. See, FIG. 2.
The area under the plasma-concentration time curve (AUC) during a dose interval at steady state (AUC_{τ, ss}).
The maximum plasma concentration at steady state (C_{max, ss}).
The time to reach maximum plasma concentration at steady state (t_{max, ss}).
Trough plasma concentration at steady state (C_{trough, ss}).

(ii) Safety

The endpoints analyzed for Period 1, Period 2, and Period 3 and follow-up period combined included discontinuations due to prostacyclin-associated AEs, adverse events (AEs) and serious adverse events (SAEs), prostacyclin-associated AEs, AEs related to injection site reactions, and PAH-related AEs.

The endpoints analyzed for (i) Period 1, Period 2, and Period 3 and follow-up period combined (ii) Period 1 and Period 2 combined, and (iii) Period 2 and Period 3 combined included discontinuations due to prostacyclin-associated AEs, discontinuations due to any AEs, AEs and SAEs, prostacyclin-associated AEs, PAH-related AEs, change from baseline in vital signs (systolic blood pressure [SBP], diastolic blood pressure [DBP], and pulse rate) and body weight, ECG abnormalities, marked laboratory abnormalities, and change from baseline in WHO FC.

### H. Statistical Methods

Approximately 20 subjects were enrolled in order to have at least 18 subjects in the i.v. Safety Set. A sample size of 18 subjects in the i.v. Safety Set would have assured, if the true rate of discontinuations due to prostacyclin-associated AEs (p0) was between 5% and 10%, that the half-width (ω) of the 95% confidence interval (CI) would be below 35%.

The statistical analysis was mainly descriptive, without formal hypothesis testing. Exact Pearson-Clopper CIs for the incidence of AEs were computed.

### I. Subject Disposition

Of 22 subjects screened, 20 were enrolled into the study. Based on the dose of oral selexipag received at the time of enrollment, 8 and 12 subjects were enrolled in the oral selexipag 200-1000 μg and 1200-1600 μg b.i.d. groups, respectively. All 20 subjects received oral selexipag in Period 1, followed by 3 i.v. infusions of selexipag in Period 2. No subject prematurely discontinued study treatment in Period 2. All subjects entered Period 3 and subsequently completed the study.

### J. Key Demographic and Baseline Data

Of the 20 subjects, 16 (80.0%) were females. Median age at baseline was 54.0 years (range: 40-75 years), with 5 subjects (25.0%)≥65 years old. Mean BMI was 28.48 kg/m² (range: 20.6-40.0 kg/m²). The study was conducted at centers in Germany and the US. All subjects were White, except 1 Asian subject.

The median time since PAH diagnosis was 88.7 months (range: 16-432 months). Based on PAH etiology, 13 subjects (65.0%) had idiopathic PAH, 1 subject had heritable PAH, and 6 subjects had PAH associated with connective tissue disease (4 subjects), congenital heart disease (1 subject), and portal hypertension (1 subject). All subjects were in WHO FC II (65.0%) or III (30.0%), except 1 subject in WHO FC I.

Of the 20 subjects, 18 were receiving two additional PAH-specific therapies (13: an ERA and a PDE-5 inhibitor, 5: an ERA and an sGC stimulator) in addition to oral selexipag at baseline. The remaining 2 subjects were receiving one additional PAH-specific therapy (1: a PDE-5 inhibitor, 1: an sGC stimulator) on top of selexipag at baseline. See, Table 5.

TABLE 5
Baseline demographics and disease characteristics

| | Total | |
|---|---|---|
| | N = 20 | |
| Sex (n [%]) | | |
| Female | 16 | (80.0) |
| Male | 4 | (20.0) |
| Age, years, mean (SD) | 56.5 | (9.43) |
| BMI, kg/m², mean (SD) | 28.48 | (6.50) |
| Country (n [%]) | | |
| Germany | 13 | (65.0) |
| USA | 7 | (35.0) |
| Time since PAH diagnosis, months, mean (SD) | 107.8 | (90.83) |

PAH etiology (n [%])

| | | |
|---|---|---|
| Idiopathic or heritable PAH | 14 | (70.0) |
| PAH associated with CTD | 4 | (20.0) |
| PAH associated with portal hypertension | 1 | (5.0) |
| PAH associated with CHD | 1 | (5.0) |

WHO functional class (FC) (n [%])

| | | |
|---|---|---|
| FC I or II | 14 | (70.0) |
| FC III | 6 | (30.0) |

Ongoing PAH-specific therapies at baseline (n [%])

| | | |
|---|---|---|
| PDE-5 inhibitors | 1 | (5.0) |
| sGC stimulators | 1 | (5.0) |
| ERA + PDE-5 inhibitor | 13 | (65.0) |
| ERA + sGC stimulator | 5 | (25.0) |

Selexipag dose at screening (n [%])

| | | |
|---|---|---|
| 200-400 μg bid | 1 | (5.0) |
| 600-1000 μg bid | 7 | (35.0) |
| 1200-1600 μg bid | 12 | (60.0) |

Ethnicity/race: all patients were white, except one (Asian).

Key inclusion criteria included male and female subjects from 18 to 75 years old inclusive, subjects with stable PAH (WHO FC I-III) and no change in PAH-specific medication and diuretics in the last 28 days prior to Visit 2, and subjects treated with selexipag at a stable dose for at least 28 days before Visit 2.

Key exclusion criteria included pregnancy or planning to be pregnant or lactating, known and documented moderate or severe hepatic impairment, subjects having received gemfibrozil at any time since initiation of selexipag, treatment with any prostacyclin and prostacyclin analogs within 28 days prior to Visit 1, a SBP<90 mmHg at Visit 1 or at Visit 2, known or suspected uncontrolled hyperthyroidism, severe renal failure and ongoing or planned dialysis, or any known factor or disease that might interfere with treatment compliance, study conduct, or interpretation of the results.

### K. Pharmacokinetic Results

For selexipag, the dose-normalized geometric mean ratios (i.v./oral selexipag) and their 90% CIs for C_{max, ss} and AUC_{τ, ss} were 1.98 (1.62, 2.43) and 2.13 (1.67, 2.70), respectively.

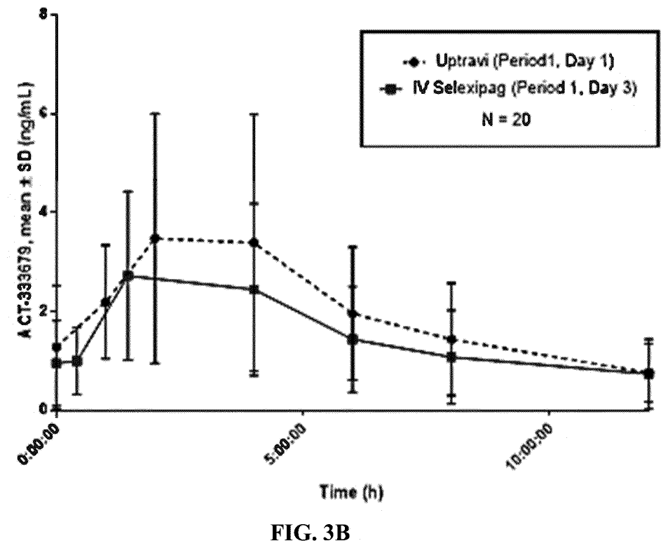

For ACT-333679, the dose-normalized geometric mean ratios (i.v./oral selexipag) and their 90% CIs for C_{max, ss} and AUC_{τ, ss} were 0.79 (0.65, 0.96) and 0.80 (0.71, 0.90), respectively. See, FIGS. 3A and 3B.

TABLE 6

| | | Selexipag | | ACT-333679 | |
|---|---|---|---|---|---|
| | | Period 1 | Period 2 | Period 1 | Period 2 |
| AUC_{τ, ss} | Geom. | 8.7 | 18.8 | 19.3 | 15.9 |
| | Mean | | | | |
| (ng · h/mL) | 95% CI | 6.5, 11.7 | 15.2, 23.2 | 13.5, 27.5 | 11.2, 22.5 |
| C_{max, ss} | Geom. | 3.9 | 7.8 | 3.3 | 2.6 |
| | Mean | | | | |
| (ng/mL) | 95% CI | 3.0, 5.1 | 6.2, 9.7 | 2.4, 4.5 | 1.9, 3.5 |

For both selexipag and ACT-333679, t_{max, ss} was comparable between the i.v. and oral selexipag periods. A dose proportional increase in exposure to selexipag and ACT-333679 was observed after i.v. administration of selexipag in the 450-1800 μg b.i.d. dose range (selexipag: slope=0.798, 90% CI: 0.387, 1.209; ACT-333679: slope=0.697, 90% CI: −0.003, 1.397). See, Table 7.

TABLE 7
Exposure to IV selexipag by infusion during Period 2

| | | | |
|---|---|---|---|
| Planned/calculated | IV infusion 1 | IV infusion 2 | IV infusion 3 |
| IV dose | (n = 20) | (n = 20) | (n = 20) |

Actual infused dose (μg)

| | | | | | | | | |
|---|---|---|---|---|---|---|---|---|
| Mean (SD) | 1372.5 | (466.86) | 1390.0 | (475.99) | 1382.2 | (476.02) | 1383.2 | (465.50) |
| Median | 1462.5 | (450-1800) | 1472.3 | (471-1980) | 1472.3 | (459-1845) | 1452.7 | (459-1845) |
| (range) | | | | | | | | |

Total time of infusion (min)

| | | | | | | | |
|---|---|---|---|---|---|---|---|
| Mean (SD) | N/A | 88.3 | (2.81) | 87.7 | (1.57) | 87.8 | (2.45) |
| Median | N/A | 87.5 | (82-95) | 88.0 | (84-91) | 88.0 | (80-91) |
| (range) | | | | | | | |

All patients completed the study treatment without interruption, except one for whom infusion was temporarily interrupted not due to an adverse event. Interruption was due to an error in setting the infusion rate.

In a post-hoc analysis on the combined AUC_{τ, ss} of selexipag plus ACT-333679 (cAUC_{τ, ss}), adjusted for their relative potencies, the dose-normalized geometric mean ratio (i.v./oral selexipag) and its 90% CI for cAUC_{τ, ss} was 0.82 (0.71, 0.93). Based on the comparable exposures to the active metabolite after oral and i.v. selexipag administration, the evaluated dosing regimen for i.v. selexipag is considered appropriate for clinical treatment of PAH patients.

### L. Safety Results

In Period 2, all 20 subjects received 3 i.v. infusions of selexipag. Subjects received the i.v. selexipag dose ranging from 450-1800 μg b.i.d. corresponding to their oral selexipag dose ranging from 400-1600 μg b.i.d. No changes in the oral selexipag doses in Period 3 compared to Period 1 were recorded. Overall (i.e., Periods 1, 2, and 3, and safety-follow-up combined), 15 subjects (75.0%) had at least 1 AE during the study. The most frequently reported AE preferred term (PT) was headache (4 subjects, 20.0%). During Period 1 (1 day; oral selexipag), 2 subjects had AEs. During i.v. selexipag infusion in Period 2 (36 hours), 10 subjects (50.0%) had at least 1 AE. See, Table 8.

TABLE 8
Number of subjects with Adverse Events per period

| | Total (low + high oral selexipag dose cohorts) | | | | |
|---|---|---|---|---|---|
| | | | | Period 3 + | |
| | Period 1 | Period 2 | | Follow-up | |
| | (oral selexipag) | (iv selexipag) | | (oral selexipag) | |
| | N = 20 | N = 20 | | N = 20 | |
| Incidence | n (%) | n (%) | | n (%) | |
| Number of subjects | 2 (10.0) | 10 | (50.0) | 8 | (40.0) |
| with at least one | | | | | |
| Adverse Event (AE) | | | | | |
| Number of subjects | 0 | 0 | | 2 | (10.0) |
| with at least one | | | | | |
| Serious adverse events | | | | | |
| (SAEs) | | | | | |
| Number of subjects | 0 | 7 | (35.0) | 1 | (5.0) |
| with at least one | | | | | |
| Prostacyclin-associated | | | | | |
| AEs | | | | | |

| | | | | |
|---|---|---|---|---|
| Number of subjects | NA | 2 | (10.0) | 0 |
| with at least one | | | | |
| Injection site reaction- | | | | |
| AE (ISR-AE) | | | | |

| | | | |
|---|---|---|---|
| Deaths | 0 | 0 | 0 |
| Discontinuations | 0 | 0 | 0 |
| due to prostacyclin- | | | |
| associated AEs | | | |

The AE PTs reported in more than 1 subject were headache (4 subjects, 20.0%) and infusion site erythema (2 subjects, 10.0%). Following re-initiation of oral selexipag in Period 3 including safety follow-up (up to 37 days), 8 subjects (40.0%) had at least 1 AE. The AE PT reported in more than 1 subject was peripheral edema (2 subjects, 10.0%), with one event reported on Day 16 and the other on Day 35. Overall (i.e., Periods 1, 2, and 3 with safety-follow-up combined), most subjects had mild (8 subjects, 40.0%) or moderate intensity AEs (5 subjects, 25.0%), with severe intensity AEs reported in 1 subject. See, Tables 9A, 9B, and 9C.

TABLE 9A
Adverse events during period 1 (24 h)

| | All adverse events (AEs) during Period 1 (Uptravi, pre-treatment) | | | |
|---|---|---|---|---|
| | Low dose | High dose | | |
| | cohort (200- | cohort (1200- | | |
| | 1000 μg) | 1600 μg) | Total | |
| | N = 8 | N = 12 | N = 20 | |
| Incidence | n (%) | n (%) | n (%) | |
| Subjects with | 1 (12.5) | 1 (8.3) | 2 | (10.0) |
| at least one | | | | |
| AE during | | | | |
| Period 1 | | | | |
| Sleep disorder | 0 | 1 (8.3) | 1 | (5.0) |
| Swelling face | 1 (12.5) | 0 | 1 | (5.0) |

TABLE 9B
Adverse events (AEs) during period 2 (36 h)

| | All adverse events (AEs during Period 2 (intravenous selexipag treatment) | | | | |
|---|---|---|---|---|---|
| | Low dose | High dose | | | |
| | cohort (200- | cohort (1200- | | | |
| | 1000 μg) | 1600 μg) | | Total | |
| | N = 8 | N = 12 | | N = 20 | |
| Incidence | n (%) | n (%) | | n (%) | |
| Subjects with | 4 (50.0) | 6 | (50.0) | 10 | (50.0) |
| at least one | | | | | |
| AE during | | | | | |
| Period 2 | | | | | |
| Headache | 1 (12.5) | 3 | (25.0) | 4 | (20.0) |
| Dysgeusia | 1 (12.5) | 0 | | 1 | (5.0) |
| Tension | 0 | 1 | (8.3) | 1 | (5.0) |
| headache | | | | | |
| Diarrhoea | 0 | 1 | (8.3) | 1 | (5.0) |
| Flatulence | 0 | 1 | (8.3) | 1 | (5.0) |
| Nausea | 0 | 1 | (8.3) | 1 | (5.0) |
| Vomiting | 0 | 1 | (8.3) | 1 | (5.0) |
| Infusion site | 0 | 2 | (16.7) | 2 | (10.0) |
| erythema | | | | | |
| Infusion site | 0 | 1 | (8.3) | 1 | (5.0) |
| swelling | | | | | |

TABLE 9C
All adverse events (AEs) during Period 3 (up
to 37 days; oral selexipag, post-treatment)

| | Low dose | High dose | | | |
|---|---|---|---|---|---|
| | cohort (200- | cohort (1200- | | | |
| | 1000 μg) | 1600 μg) | | Total | |
| | N = 8 | N = 12 | | N = 20 | |
| Incidence | n (%) | n (%) | | n (%) | |
| Subjects with | 2 (25.0) | 6 | (50.0) | 8 | (40.0) |
| at least one | | | | | |
| AE during | | | | | |
| Period 3 | | | | | |
| Oedema peripheral | 1 (12.5) | 1 | (8.3) | 2 | (10.0) |
| Nasopharyngitis | 1 (12.5) | 0 | | 1 | (5.0) |
| Upper respiratory | 0 | 1 | (8.3) | 1 | (5.0) |
| tract infection | | | | | |
| Right ventricular | 0 | 1 | (8.3) | 1 | (5.0) |
| failure | | | | | |
| Blindness | 0 | 1 | (8.3) | 1 | (5.0) |
| unilateral | | | | | |
| Rhegmatogenous | 0 | 1 | (8.3) | 1 | (5.0) |
| retinal detachment | | | | | |
| Nausea | 0 | 1 | (8.3) | 1 | (5.0) |
| Hypokalaemia | 0 | 1 | (8.3) | 1 | (5.0) |
| Headache | 0 | 1 | (8.3) | 1 | (5.0) |
| Chromaturia | 0 | 1 | (8.3) | 1 | (5.0) |
| Cough | 0 | 1 | (8.3) | 1 | (5.0) |
| Dyspnoea | 0 | 1 | (8.3) | 1 | (5.0) |
| Flushing | 0 | 1 | (8.3) | 1 | (5.0) |

No subject died or discontinued study treatment due to an AE. SAEs were reported for 2 subjects in Period 3 (including safety follow-up) of the study. One elderly subject with a medical history of bilateral cataract surgery and ongoing type 2 diabetes mellitus and arterial hypertension developed rhegmatogenous retinal detachment with unilateral blindness one day after i.v. selexipag infusion 3 (1800 μg). Vision reportedly improved to 80% under treatment. 1 subject had an SAE of right ventricular failure which occurred in the context of a respiratory tract infection. The SAE resolved without sequelae. For both subjects treatment with selexipag remained ongoing. These SAEs are confounded by medical history (retinal detachment/unilateral blindness) and concurrent events (RVF). See, Tables 10 and 11.

TABLE 10
All adverse events (AEs) during Period
1 (oral selexipag, pre-treatment)

| | Low dose, | High dose | | | |
|---|---|---|---|---|---|
| | cohort (200- | cohort (1200- | | | |
| | 1000 μg) | 1600 μg) | | Total | |
| | N = 8 | N = 12 | | N = 20 | |
| Incidence | n (%) | n (%) | | n (%) | |
| Subjects with | 1 (12.5) | 1 | (8.3) | 2 | (10.0) |
| at least one | | | | | |
| AE during Period 1 | | | | | |
| Sleep disorder | 0 | 1 | (8.3) | 1 | (5.0) |

| | | | | |
|---|---|---|---|---|
| Swelling face | 1 (12.5) | 0 | 1 | (5.0) |

All adverse events (AEs during Period 2 (intravenous selexipag treatment)

| Subjects with | 4 (50.0) | 6 | (50.0) | 10 | (50.0) |
|---|---|---|---|---|---|
| at least one | | | | | |
| AE during | | | | | |
| Period 2 | | | | | |
| Headache | 1 (12.5) | 3 | (25.0) | 4 | (20.0) |
| Dysgeusia | 1 (12.5) | 0 | | 1 | (5.0) |
| Tension | 0 | 1 | (8.3) | 1 | (5.0) |
| headache | | | | | |
| Diarrhoea | 0 | 1 | (8.3) | 1 | (5.0) |
| Flatulence | 0 | 1 | (8.3) | 1 | (5.0) |
| Nausea | 0 | 1 | (8.3) | 1 | (5.0) |
| Vomiting | 0 | 1 | (8.3) | 1 | (5.0) |
| Infusion site | 0 | 2 | (16.7) | 2 | (10.0) |
| erythema | | | | | |
| Infusion site | 0 | 1 | (8.3) | 1 | (5.0) |
| swelling | | | | | |

All adverse events (AEs) during Period 2 (intravenous selexipag treatment)

| Incorrect drug | 1 (12.5) | 0 | | 1 | (5.0) |
|---|---|---|---|---|---|
| administration | | | | | |
| rate | | | | | |
| Vascular access | 0 | 1 | (8.3) | 1 | (5.0) |
| complication | | | | | |
| Epistaxis | 1 (12.5) | 0 | | 1 | (5.0) |
| Nasal congestion | 0 | 1 | (8.3) | 1 | (5.0) |
| Lymphadenopathy | 1 (12.5) | 0 | | 1 | (5.0) |
| Tachycardia | 1 (12.5) | 0 | | 1 | (5.0) |
| Myalgia | 0 | 1 | (8.3) | 1 | (5.0) |
| Pain in jaw | 0 | 1 | (8.3) | 1 | (5.0) |
| Flushing | 0 | 1 | (8.3) | 1 | (5.0) |

All adverse events (AEs) during Period 3 (oral selexipag, post-treatment)

| Subjects with | 2 (25.0) | 6 | (50.0) | 8 | (40.0) |
|---|---|---|---|---|---|
| at least one | | | | | |
| AE during | | | | | |
| Period 3 | | | | | |
| Oedema peripheral | 1 (12.5) | 1 | (8.3) | 2 | (10.0) |
| Nasopharyngitis | 1 (12.5) | 0 | | 1 | (5.0) |
| Upper respiratory | 0 | 1 | (8.3) | 1 | (5.0) |
| tract infection | | | | | |
| Right ventricular | 0 | 1 | (8.3) | 1 | (5.0) |
| failure | | | | | |
| Blindness | 0 | 1 | (8.3) | 1 | (5.0) |
| unilateral | | | | | |
| Rhegmatogenous | 0 | 1 | (8.3) | 1 | (5.0) |
| retinal | | | | | |
| detachment | | | | | |
| Nausea | 0 | 1 | (8.3) | 1 | (5.0) |
| Hypokalaemia | 0 | 1 | (8.3) | 1 | (5.0) |
| Headache | 0 | 1 | (8.3) | 1 | (5.0) |
| Chromaturia | 0 | 1 | (8.3) | 1 | (5.0) |
| Cough | 0 | 1 | (8.3) | 1 | (5.0) |
| Dyspnoea | 0 | 1 | (8.3) | 1 | (5.0) |
| Flushing | 0 | 1 | (8.3) | 1 | (5.0) |

TABLE 11
Serious adverse events (SAEs) per Preferred term (PT) and period

| | Total (low + high oral selexipag dose cohorts) | | | |
|---|---|---|---|---|
| | | | Period 3 (oral | |
| | Period 1 (oral | Period 2 | selexipag) + | |
| | selexipag) | (iv selexipag) | Follow-up | |
| | N = 20 | N = 20 | N = 20 | |
| Incidence | n (%)* | n (%)* | n (%)* | |
| Patients with | 0 | 0 | 2 | (10.0) |
| at least 1 SAE | | | | |
| Blindness | 0 | 0 | 1 | (5.0) |
| unilateral ¹ | | | | |
| Rhegmatogenous | 0 | 0 | 1 | (5.0) |
| retinal | | | | |
| detachment ¹ | | | | |
| Right ventricular | 0 | 0 | 1 | (5.0) |
| failure ² | | | | |

No PAH-related AEs were reported in Periods 1 and 2. 3 subjects had PAH-related AEs in Period 3, peripheral edema (2 subjects) and right ventricular failure in the context of respiratory tract infection (1 subject; described above). During Period 2, 7 subjects (35.0%) had at least 1 prostacyclin-associated AE, with headache reported in 4 subjects. All prostacyclin-associated AEs in Period 2 were considered by the investigator to be study treatment-related, except 1 AE of tension headache. None of the prostacyclin-associated AEs were reported as severe, serious, or resulted in discontinuation of study treatment. See, Table 12.

TABLE 12
Prostacyclin-associated AEs during Periods 1-3
Prostacyclin-associated AEs during Period 1 (oral selexipag)

| | Low dose | High dose | |
|---|---|---|---|
| | cohort (200- | cohort (1200- | |
| | 1000 μg) | 1600 μg) | Total |
| | N = 8 | N = 12 | N = 20 |
| Incidence | n (%) | n (%) | n (%) |
| Subjects with | 0 | 0 | 0 |
| at least on AE | | | |

Prostacyclin-associated AEs during Period 2 (intravenous selexipag treatment)

| | | | | | |
|---|---|---|---|---|---|
| Subjects with | 1 (12.5) | 6 | (50.0) | 7 | (35.0) |
| at least on AE | | | | | |
| Headache | 1 (12.5) | 3 | (25.0) | 4 | (20.0) |
| Diarrhoea | 0 | 1 | (8.3) | 1 | (5.0) |
| Flushing | 0 | 1 | (8.3) | 1 | (5.0) |
| Myalgia | 0 | 1 | (8.3) | 1 | (5.0) |
| Nausea | 0 | 1 | (8.3) | 1 | (5.0) |
| Pain in jaw | 0 | 1 | (8.3) | 1 | (5.0) |
| Tension headache | 0 | 1 | (8.3) | 1 | (5.0) |
| Vomiting | 0 | 1 | (8.3) | 1 | (5.0) |

Prostacyclin-associated AEs during Period 3 (intravenous selexipag treatment)

| | | | | | |
|---|---|---|---|---|---|
| Flushing | 0 | 1 | (8.3) | 1 | (5.0) |
| Headache | 0 | 1 | (8.3) | 1 | (5.0) |
| Nausea | 0 | 1 | (8.3) | 1 | (5.0) |

During Period 2, two subjects had clinically significant injection site reactions as determined by the investigator. These were mild intensity infusion site erythema and swelling (1 subject) and mild-to-moderate infusion site erythema (1 subject). The clinically significant injection site reactions were considered by the investigator to be study-treatment-related. For hemoglobin, the mean change from baseline (129.4 g/L) to Visit 2 Day 3 was −5.7 g/L. At Visit 3 (Day 12±2 days), the mean change was 1.2 g/L. Changes from baseline in other hematology and clinical chemistry variables were unremarkable. No changes in systolic blood pressure of >40 mmHg and no AE of hypotension were reported for any subject. See, Tables 13 and 14.

TABLE 13
Blood pressure abnormalities per period

| | Total (low + high oral selexipag dose cohorts) | | |
|---|---|---|---|
| | | | Period 3 (oral |
| | Period 1 (oral | Period 2 | selexipag) + |
| | selexipag) | (iv selexipag) | Follow-up |
| | N = 20 | N = 20 | N = 20 |
| Incidence | n (%) | n (%) | n (%) |

Systolic blood pressure (SBP, in mmHg)

| | | | | |
|---|---|---|---|---|
| SBP < 90 | 0 | 2 | (10.0) | 0 |
| SBP decrease | 0 | 2 | (10.0) | 0 |
| from baseline > 40 | | | | |
| SBP ≥ 160 | 0 | 1 | (5.0) | 0 |

Diastolic blood pressure (DBP, in mmHg)

| | | | | |
|---|---|---|---|---|
| DBP < 50 | 0 | 5 | (25.0) | 0 |
| DBP decrease | 1 (5.0) | 3 | (15.0) | 2 (10.0) |
| from baseline > 20 | | | | |

| | | | |
|---|---|---|---|
| DBP ≥ 100 | 0 | 0 | 0 |

TABLE 14
Blood pressure abnormalities during Period 2

| | Low dose | High dose | |
|---|---|---|---|
| | cohort (200- | cohort (1200- | |
| | 1000 μg) | 1600 μg) | Total |
| | N = 8 | N = 12 | N = 20 |
| Incidence | n (%) | n (%) | n (%) |

Systolic blood pressure (SBP, in mmHg)

| | | | | | |
|---|---|---|---|---|---|
| SBP < 90 | 1 (12.5) | 1 | (8.3) | 2 | (10.0) |
| SBP decrease | 1 (12.5) | 1 | (8.3) | 2 | (10.0) |
| from baseline > 40 | | | | | |
| SBP ≥ 160 | 0 | 1 | (8.3) | 1 | (5.0) |

Diastolic blood pressure (DBP, in mmHg)

| | | | | | |
|---|---|---|---|---|---|
| DBP < 50 | 2 (25.0) | 3 | (25.0) | 5 | (25.0) |
| DBP decrease | 1 (12.5) | 2 | (16.7) | 3 | (15.0) |
| from baseline > 20 | | | | | |

| | | | |
|---|---|---|---|
| DBP ≥ 100 | 0 | 0 | 0 |

During Period 2, mean changes from pre- to post-infusion in heart rate were 2.1, 0.1, and 3.5 bpm following infusions 1, 2, and 3, respectively. During Period 2, QT_{c}F prolongation to >450 ms was reported for 3 subjects (15.8%) and QT_{c}F prolongation to >480 ms was reported for 4 subjects (21.1%). Of these 7 subjects, 6 already had QT_{c}F prolongation to >450 ms in Period 1 (i.e., at Visit 2 Day 2 prior to the start of i.v. infusion of selexipag). No changes in WHO FC of any subject were reported during the study.

### M. Conclusions

Exposure to the active metabolite of selexipag, ACT-333679, was comparable after i.v. and oral selexipag administration. Further the switch from oral selexipag (Period 1) to i.v. selexipag (Period 2) and back to oral selexipag (Period 3) was well tolerated. The AE profile was consistent with the known safety profile of selexipag, with no unexpected safety findings observed during treatment with i.v. selexipag.

1-23. (canceled)

24. A method of avoiding treatment interruption in a patient receiving an oral dose of selexipag for treating pulmonary arterial hypertension, wherein the patient is temporarily unable to take oral medication, comprising administering to the patient an intravenous (IV) dose of selexipag.

25. The method of claim 24, wherein the patient receives the oral dose of selexipag for at least about 28 days prior to administration of the IV dose.

26. The method of claim 24, wherein the patient did not receive any prostacyclin or prostacyclin analogs, other than selexipag, for at least about 28 days prior to administration of the IV dose.

27. The method of claim 24, wherein the patient has a systolic blood pressure of less than about 90 mmHg prior to administration of the IV dose.

28. The method of claim 24, wherein the oral dose of selexipag comprises from about 200 μg to about 1600 μg twice daily.

29. The method of claim 28, wherein the oral dose of selexipag comprises about 200 μg to about 1000 μg twice daily.

30. The method of claim 28, wherein the oral dose of selexipag comprises about 1200 μg to about 1600 μg twice daily.

31. The method of claim 24, wherein the IV dose is selected to achieve a comparable exposure to ACT-333679 when compared to the oral dose.

32. The method of claim 31, wherein the IV dose to oral dose ratio is about 1.125.

33. The method of claim 24, wherein, following administration of the IV dose, the patient resumes the same oral dose as prescribed before the IV dose administration.

34. The method of claim 24, wherein a first period of treatment comprises the administration of the oral dose, a second period comprises the administration of the IV dose, and a third period comprises resumption of the same oral dose as administered in the first period.

35. The method of claim 34, wherein area under the plasma-concentration time curve during a dose interval at steady state of selexipag is about two times greater in the second period than in the first period.

36. The method of claim 34, wherein area under the plasma-concentration time curve during a dose interval at steady state of ACT-333679 is comparable in the first and second period.

37. The method of claim 34, wherein time to reach maximum plasma concentration at steady state of selexipag and ACT-333679 is comparable in the first and second period.

38. The method of claim 34, wherein the first period comprises the oral dose twice daily, once before mid-day and once after mid-day.

39. The method of claim 38, wherein the second period comprises three infusions of the IV dose.

40. The method of claim 39, wherein each infusion is from about 80 to about 90 minutes in duration.

41. The method of claim 40, wherein two of the infusions are administered in one day, once before mid-day and once after mid-day.

42. The method of claim 40, wherein the third infusion is administered on the next day following the second infusion.

43. The method of claim 42, wherein the third period comprises resumption of the oral dose on the same day of, and following, the third infusion.

44. The method of claim 24, wherein the IV dose of selexipag comprises about 225 μg to about 1800 μg twice daily.

45. The method of claim 24, wherein the oral dose is in the form of a tablet.

46. The method of claim 24, wherein the patient is temporarily unable to take oral medication due to a hospitalization.
